(19) United States
(12) Patent Application Publication
(10) Pub. No.: US 2019/0165488 A1
(43) Pub. Date: May 30, 2019

(54) DUAL CIRCULAR POLARIZATION DIVERSITY SCHEME FOR MICROWAVE LINK

(71) Applicant: T-Mobile USA, Inc., Bellevue, WA (US)

(72) Inventor: Chad Au, Kirkland, WA (US)

(21) Appl. No.: 15/827,449

(22) Filed: Nov. 30, 2017

(51) Int. Cl. H01Q 25/00; H01Q 21/26; H01Q 1/24; H01Q 9/26; H04B 7/10; A61N 5/02

(52) U.S. Cl. CPC H01Q 25/001; H01Q 21/26; A61N 5/02; H01Q 9/26; H04B 7/10; H01Q 1/246

(57) ABSTRACT

A dual circular polarization diversity scheme reduces the effects of atmospheric fading while providing a more efficient implementation and a more robust wireless link in a telecommunications backhaul network. A circular polarization diversity scheme alleviates issues with misalignment of transmitter and receiver polarization and thereby improves performance. By controlling the feed into the transmit antenna, the output can be a dual circular polarization scheme that simultaneously outputs left-hand circular and right-hand circular polarized radio signal.

### BACKGROUND

Current microwave links are designed to a reliability of 99.999%, meaning that in a year's time, there can only be a total outage of no more than 5 minutes. To meet that standard, atmospheric factors such as temperature, humidity, and precipitation must be accounted for since atmospheric conditions can cause the received signal level (RSL) to fade and drop below the receiver sensitivity threshold and impact reliability.

Microwave parabolic reflector antennas used currently in a backhaul network are typically linearly polarized: either vertical or horizontal, as determined by its feed at the focal point. For a microwave link without space diversity, only one antenna is installed at each end of the link, which is highly affected by the effects of fading.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 illustrates an overview of a system for providing a dual circular polarization diversity scheme.

FIG. 2 illustrates a diversity antenna scheme based on space diversity.

FIG. 3A illustrates a linear vertical polarization propagating wave.

FIG. 3B illustrates a liner horizontal polarization propagating wave.

FIG. 4 illustrates a dual dipole antenna system that can be driven to provide circular polarization of an electromagnetic wave.

FIG. 5 illustrates an example patch antenna with a dual circularly polarized element configured to be driven with a dual circular polarization diversity scheme.

FIG. 6 is an example illustration of circular polarization of an electromagnetic wave, illustrating how the electric field of the wave has a constant magnitude but a rotating electric field vector over time.

FIG. 7 is an illustration showing a circular polarization of an E-field sequence over time.

### DETAILED DESCRIPTION

This disclosure describes, in part, systems and methods for providing a dual circular polarization diversity scheme. In some implementations, the antenna system is used in a microwave link, such as, for example, a backhaul network where directional transceivers require a high degree of robustness. In general, a microwave link used a radio signal within the spectral band of about 300 MHz to 300 GHz, and having a wavelength of about 1 m to 1 mm, respectively.

The backhaul portion of a network is typically responsible for providing the intermediate links between the core network and the smaller subnets. In many cases, the backhaul network includes cell towers that provide wireless service to a subnet and a direct link to the rest of the provider's network, and are responsible for providing a connection to the internet. In some instances, the backhaul network includes multiple towers that are capable of operating together to provide point-to-point, or point-to-multipoint connection links over high-capacity radio links. A wireless backhaul, including a multi-hop wireless architecture, can be used to provide service in remote areas, create efficient large coverage areas, and expand with growing demand in emerging markets.

Current microwave links, such as those used in a wireless backhaul network, are sensitive to atmospheric factors, such as temperature, humidity, and precipitation which can cause the received signal level (“RSL”) to fade and drop below the receiver sensitivity threshold, which can impact reliability of the link. While these factors can be cumulatively quantified and addressed in the antenna design by including a fade margin, current implementations are complicated, require duplicate antenna hardware, and increase the static loading and wind loading on a tower.

In wireless communication, fading is an attenuation of the signal based upon time, geographical position, and/or environmental factors. Of particular concern in microwave radio transmissions is the effect of rain fade, which is the absorption of a microwave radio frequency signal by atmospheric rain, snow, or ice. Similarly, the electromagnetic interference caused by the leading edge of a storm front can also be responsible for rain fade.

One way of addressing fading is by implementing a space and/or frequency diversity to the signal. Currently, microwave parabolic reflector antennas are linearly polarized, either vertically or horizontally, as determined by the feed at the focal point. For a microwave link without space diversity, a single antenna is installed at each end of the link which is highly affected by the effects of fading. Through a diversity arrangement, fading degradation can be reduced by introducing two de-correlated radio frequency paths.

In a space-diversity scheme, a second antenna can be added at a sufficiently spaced location on the tower or structure from an existing antenna. The result is dual antennas spaced a distance apart both relaying the radio frequency signal. However, difficulties arise with space diversity because of the increase in hardware necessary to implement this scheme. For instance, additional space must be allocated on a tower, which must be able to withstand the additional static load and wind load from the additional antenna structure. Additionally, the radio frequency paths of the antennas should clear the Fresnel zone in order to avoid blockage of the radio frequency paths due to terrain.

The Fresnel zone is defined by the phase shift that occurs when a radio frequency wave deflects off an object within the transmit path. The result is that a radio frequency wave that follows a path through the Fresnel zone may arrive at the receiver out of sync with a line of sight radio frequency wave. The deflected signal can result in destructive interference which may further cause a RSL at the receiver that is below an acceptable threshold.

The polarization of the signal can influence how the signal is received at the receiver. For example, where a radio frequency signal is linearly polarized in a vertical direction and it deflects of a horizontal object within the first Fresnel zone, such as the ground, before being received at a receiving antenna, the arriving signal will be 180 degrees out of phase relative to the transmitted signal. This out of phase signal arrival will destructively interfere with the line of sight signal received at the receiving antenna, thus degrading the overall strength of the received signal.

According to an embodiment, a backhaul network communications link is established with a first parabolic antenna configured to transmit a right-hand circular polarized radio signal at a frequency as well as a left-hand circular polarized radio signal at the same frequency. A second parabolic antenna can be configured to receive the right-hand circular polarized radio signal at the frequency and the left-hand circular polarized radio signal at the frequency. In other words, two antennas, using orthogonal circularly polarized signals can create a communications link that uses a diversity scheme. In some cases, the first parabolic antenna is a patch antenna that has two feed points, the parabolic antenna may also be a pair of dipole antennas. When driven out of phase, a pair of dipole antennas can create a circularly polarized signal. The antenna may transmit a signal having a frequency within the microwave portion of the electromagnetic spectrum.

In some cases, a polarized diversity scheme antenna system can be used in a telecommunications backhaul network. The system may include a first antenna having a first aperture. An antenna element can be configured to broadcast a first circularly polarized signal through the aperture. The antenna element may be further configured to broadcast a second circularly polarized signal through the aperture. The second circularly polarized signal can have an orthogonal direction of polarization to the first circularly polarized signal. In other words, a single antenna can transmit a dual circularly polarized signal through a single antenna aperture.

The first antenna element may include a dielectric substrate and a conductive patch disposed on the dielectric substrate. In some cases, the conductive patch may be rectangular and have four edges. In some cases, the conductive patch is square, or nearly square. The conductive patch may have a slot formed along at least a portion of its diagonal. A feed point may be located on a first edge of the conductive patch for feeding an input signal to the conductive patch. In some cases, a second feed point may be located on a second edge of the conductive patch, the second edge being orthogonal to the first edge.

When used in a communications link, a second antenna may include a second antenna element for receiving the first circularly polarized signal and the second circularly polarized signal sent from the first antenna.

In a patch antenna, the conductive patch may have a length that is within the range of from about 50 cm to 0.05 cm (approximately ½ wavelength), which allows it to transmit at a frequency within the microwave portion of the electromagnetic spectrum. For higher gain performance multiple patches may be arranged in an array for the feed.

According to other examples, a microwave telecommunications link includes a first antenna mounted stationary and having a reflective dish, a first antenna element that broadcasts a first circularly polarized signal and a second circularly polarized signal, the second circularly polarized signal orthogonal to the first circularly polarized signal. A second antenna may likewise have a reflective dish and a second antenna element that receives the first circularly polarized signal and the second circularly polarized signal.

In some cases, one of the antennas in the link is on a moveable platform, such as, for example, a satellite, an aircraft, a ship, a train, or some other passenger vehicle.

In some examples, the first antenna is a patch antenna having two input feeds. The patch antenna may include a conductive patch having a length and a width that are substantially equal. The two input feeds may be connected to the patch antenna at a location approximately midway along the length, and approximately midway along the width. The length and width may be within the range of from about 50 cm to 0.05 cm.

In some cases, the first antenna further comprises an antenna aperture and the first circularly polarized signal and the second circularly polarized signal are broadcast through the antenna aperture. Thus, an antenna may broadcast both a right-hand circularly polarized signal and a left-hand circularly polarized signal through a common aperture.

In some cases, the antenna is a patch antenna having a single feed and symmetric perturbation elements. For example, a patch antenna may include symmetric grooves, ridges, slots, or some other geometric perturbation element for influencing the output signal. In some cases, a single feed may be applied to the patch antenna, and through the design of perturbation elements, a circularly polarized signal may be transmitted, or in some cases, a dual circularly polarized signal. In some cases, the antenna is a printed circular ring patch antenna. In some cases, the antenna broadcasts at least two de-correlated signals through a common antenna aperture.

These, and other features, will be further described by reference to the attached figures.

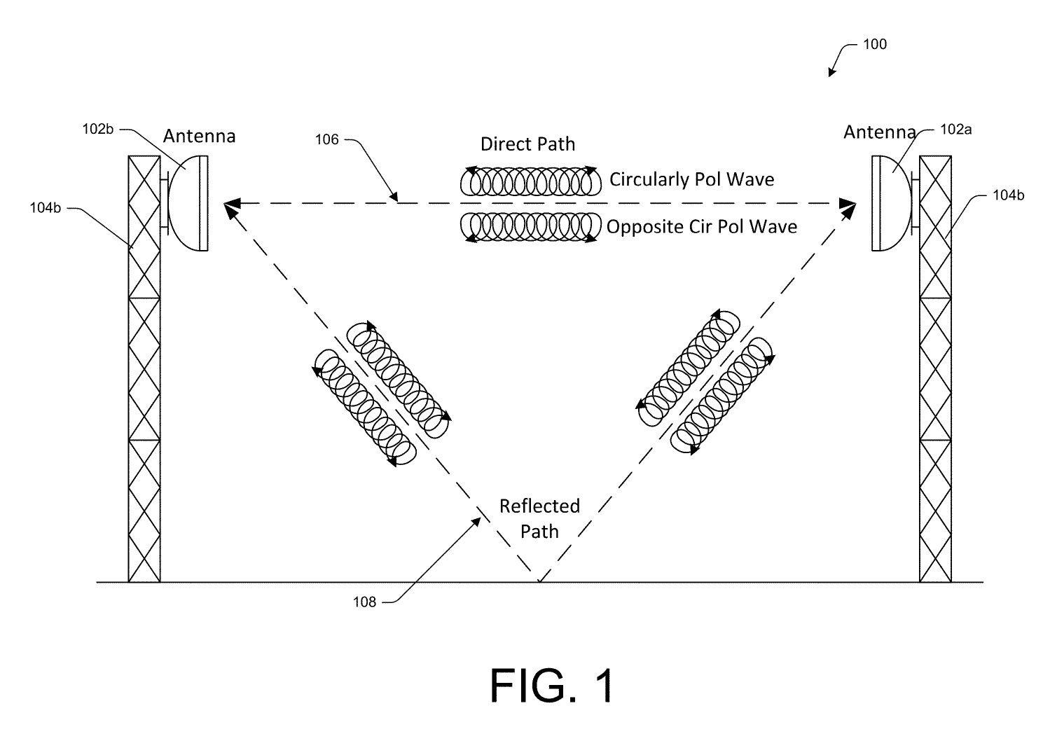

FIG. 1 illustrates an overview of a system 100 for providing a dual circular polarization diversity scheme. The system 100 may include one or more antennas 102a, 102b mounted to a structure 104a, 104b. The antennas 102a, 102b may be mounted to any suitable structure, and may be mounted on the ground, on a building, a tower, or on a moveable platform, such as a satellite or aircraft.

The system may provide for a link between the antennas 102a, 102b such as a microwave link to transmit and receive data, such as part of a telecommunications backhaul network. The antennas may be positioned such that there is a line of sight transmission path 106 between the antennas 102a 102b. In addition, a reflected path 108 may exist between the antennas to provide transmission path diversity. Of course, while a link between two antennas is shown, a wireless communication network may include numerous antennas that form point-to-point or point-to-multipoint communication links. Furthermore, the wireless communication network may include satellites as part of the network for relaying radio signals.

As illustrated, each antenna 102a, 102b may be configured to transmit a radio frequency wave with a circular polarization scheme. In some instances, both a right-hand circular polarization and a left-hand circular polarization signals are transmitted to provide highly decorrelated propagation and antenna responses. Circular polarization is more resistant to signal degrading due to atmospheric fading effects than is linear polarization. Moreover, reception of circularly polarized signal is not impacted by the Faraday Effect.

The Faraday Effect is a magneto-optical phenomenon resulting in a rotation of the plane of polarization that is linearly proportional to the component of the magnetic field in the propagation direction. This effect varies as atmospheric conditions change throughout the day due to temperature fluctuations and the amount of moisture in the air. A rotation of a radio frequency signal that is linearly polarized causes a reduced RSL due to the polarization being misaligned with the receiver.

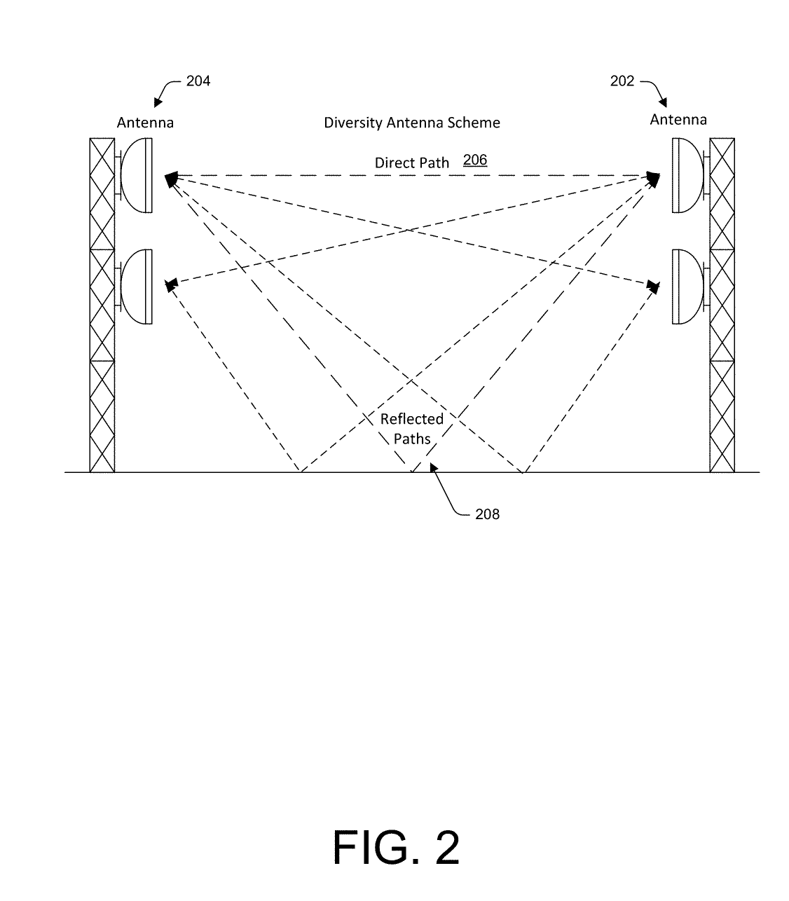

FIG. 2 illustrates a spatial diversity scheme in which two antennas are located on each tower at each end of the link. A first pair of antennas 202 communicate with a second pair of antennas 204. The antennas provide for distinct decorrelated paths having both a direct path 206 and a reflected path 208. However, where the antennas utilize a linear polarization, the transmission paths should be designed to clear the Fresnel Zone to avoid blockage of the RF path due to terrain, which results in signal degradation.

In many cases, radio frequency carriers of different frequencies are transmitted and received through an antenna at each end so as to have a diverse fading profile for each carrier such that the link for one frequency is potentially less impacted by fading than the other frequency carrier. Thus, in addition to space diversity, frequency diversity may also be implemented to maintain a robust communications link. However, this is spectrally inefficient since more spectrum is require to be used to support this redundant scheme, and the additional spectrum may not be readily available.

Furthermore, as described, the illustrated space diversity scheme requires at least two antennas to be installed at each end of the link, which increases equipment costs, tower space, and may be prohibitive if the structure is not able to support the extra loading necessitated by the additional antenna structure.

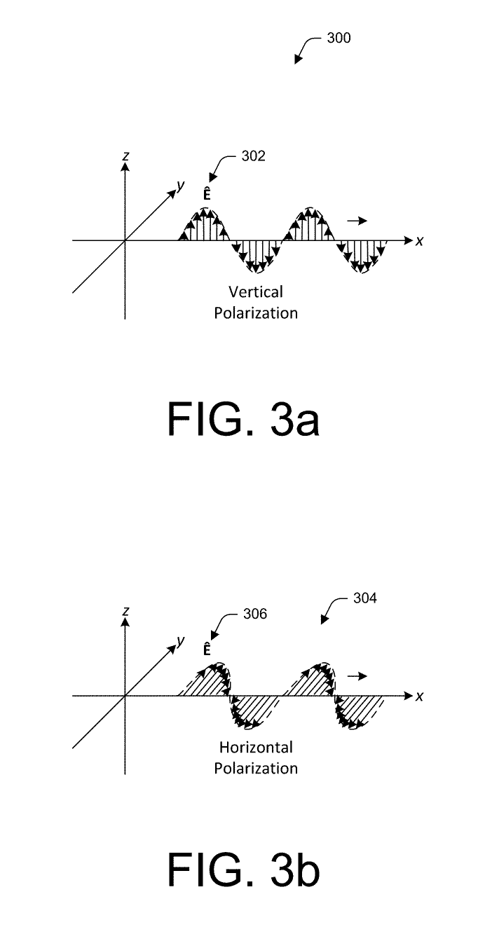

FIG. 3a is an illustration of a radio frequency signal exhibiting vertical linear polarization 300. The dominant electric field vector (Ê) 302 in an electromagnetic field defines the polarization. For vertical liner polarization, the Ê 302 oscillates in the x-z plane with changing phase. For horizontal linear polarization 304, as shown in FIG. 3b, the Ê 306 oscillates in the x-y plane with changing phase.

A linearly polarized antenna requires that it be aligned with the dominant electric field impinging upon it in order to be co-polarized in order to effectively capture the signal. The RSL will degrade proportionally to the amount of misalignment. An electromagnetic field consists of the dominant component as well as electric field vectors in other orientations. Misalignment with the dominant component may also cause the antenna to capture some of the other polarized components, which can result in an increase in signal interference since the components can combine destructively with the dominant E.

By contrast, circular polarization is more resistant to signal degradation due to atmospheric fading effects. Furthermore, a circular polarized signal is not markedly impacted by the Faraday Effect. A diversity scheme can be established by transmitting a dual circular polarization scheme in which both a right-handed circularly polarized signal and a left-handed circularly polarized signal are transmitted along the same signal path. A right-hand circular (RHC) signal is generally orthogonal to a left-hand circular (LHC) signal. Therefore, their propagation and antenna response are highly decorrelated, which can provide a robust communications link that overcomes many of the noted issues experienced by current antenna links that rely on linear polarization, space diversity, and/or frequency diversity.

A dual circularly polarized antenna may be a parabolic dish antenna having a single antenna aperture and the diversity scheme is applied by the input signal to the antenna.

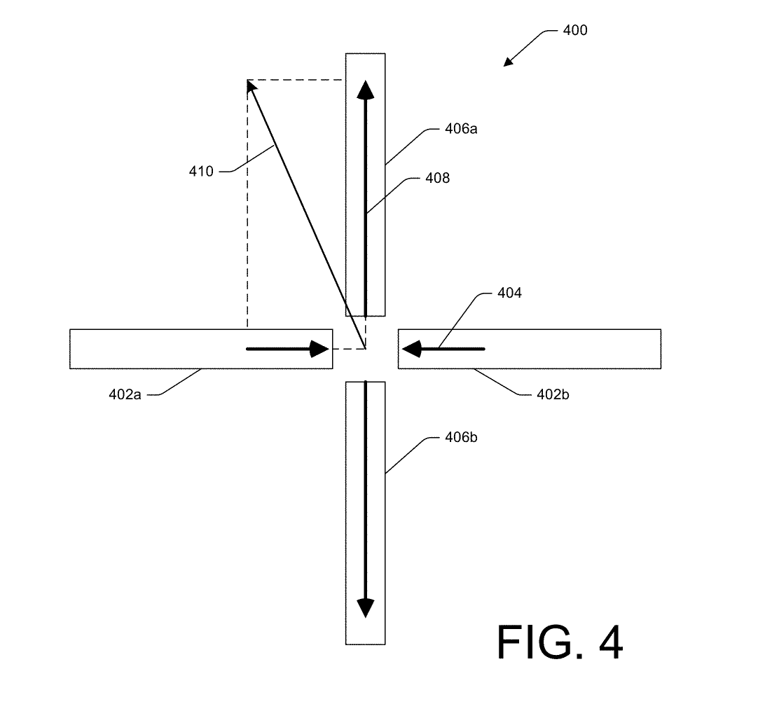

FIG. 4 illustrates one embodiment of a dual circularly polarized antenna that utilizes two phase coordinated linear polarized dipole antennas in combination. A first pair of antenna elements 402a, 402b operates in a horizontal linear polarization. They produce an electric field vector in a horizontal x-y plane 404. A second pair of antenna elements 406a, 406b produce a vertical electrical field vector in a vertical x-z plane 408. The horizontal electrical field vector 404 combines with the vertical electric field vector 408 to produce a circular electrical field vector 410. That is, the circular electric field vector 410 is a combination of the magnitudes and phases of the horizontal electric field vector 404 and the vertical electric field vector 408. As the two linear vectors 404, 408 are driven with a sine wave input, their magnitudes increase to a maximum and decrease to a minimum according to the input signal. Where the first pair of antenna elements 402a, 402b are driven ninety degrees out of phase with the second pair of antenna elements 406a, 406b, the resulting combination produces a circular electric field vector 410 that maintains a constant magnitude but the plane of polarization rotates in a corkscrew pattern making a complete revolution during each wavelength. Thus, the energy will be radiated in both the horizontal and vertical planes, and also every plane in between.

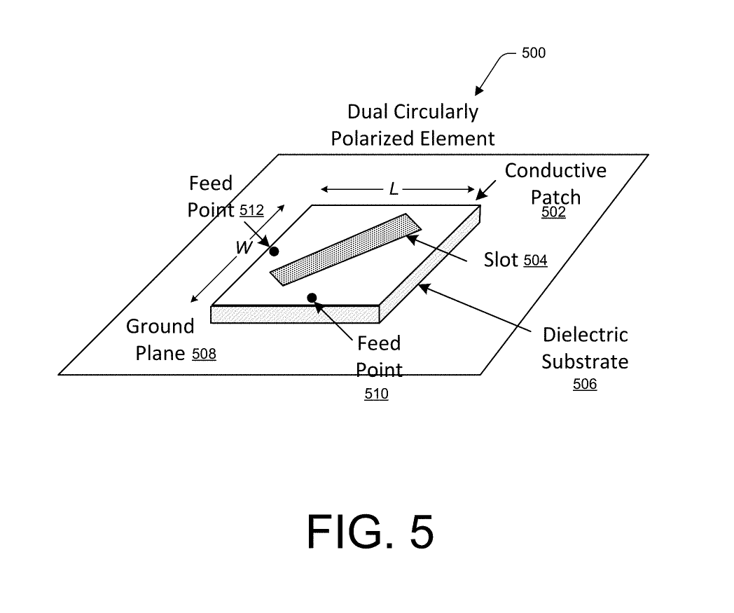

FIG. 5 illustrates another embodiment of a dual circularly polarized element 500 utilizing a conductive patch 502, which may be used as part of a parabolic antenna system. The antenna element 500 includes a conductive patch 502 that may be formed of any suitable conductive material, such as, for example, copper. The conductive patch 502 may be generally rectangular or, in some cases, square, having a length L and a width W and is bonded to a dielectric substrate 506, which has permittivity εᵣ. The dielectric substrate 506 is in intimate contact with a ground plane 508. The ground plane 508 may be formed of a conductive material, which may be the same material as the conductive patch 502, or may be another conductive material. The antenna element 500 can be configured to transmit a signal that is dual circularly polarized based upon the geometry of the patch and the input feeds. In some instances, the patch 502 has a slot 504 formed in the conductive patch 502 that is generally along a diagonal of the rectangular or square conductive patch 502. A first feed point 510 inputs a signal to the conductive patch 502 along a first edge of the patch 502 and a second feed point 512 inputs a signal along a second edge of the patch 502. As illustrated, the second feed point 512 may be located on an edge that is orthogonal to the edge where the first feed point 510 connects.

As the conductive patch 502 resonates from an input provided at the first feed point 510, the slot 504 will cause the transmission to be circularly polarized. In the embodiment illustrated in FIG. 5, an input entering at the first feed point 510 will be right-hand circularly polarized based upon the orientation of the slot 504 in the conductive patch 502. In some instances, an input will also be provided at the second feed point 512, which will result in a left-hand circularly polarized signal based upon the orientation of the slot 504 in relation to the second feed point 512.

The frequency of the transmitted electromagnetic signal entering at the second feed point 512 is determined by the length L of the patch. Specifically, the center frequency of the transmission can be approximated by equation 1 below.

$${f}_{c} \approx \frac{c}{2 L \sqrt{{ɛ}_{r}}} = \frac{1}{2 L \sqrt{{ɛ}_{0} {ɛ}_{r} {μ}_{0}}} \qquad Equ ( 1 )$$

According to equation 1, the patch 502 should be designed to have a length that is about equal to one half of a wavelength within the dielectric substrate 506. Similarly, in the case of a dual circularly polarized signal, the width of the patch 502 should also be about equal to one half of a wavelength within the dielectric substrate 506. Accordingly, where the conductive patch 502 is formed as a square, the transmission caused by the first feed point 510 and the second feed point 512 will be roughly the same frequency having orthogonal circular polarization directions.

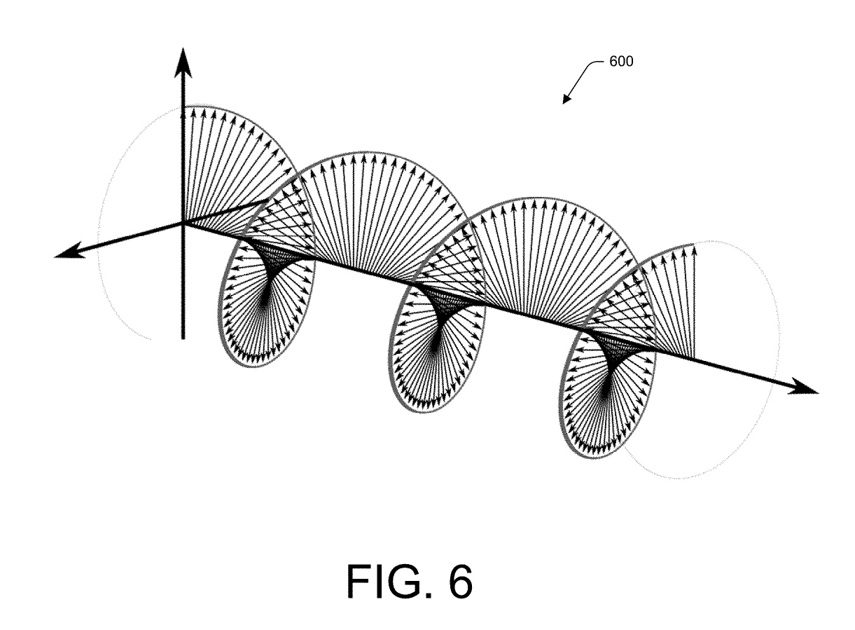

FIG. 6 illustrates a circularly polarized signal 600. The circularly polarized signal has a constant magnitude while the plane of polarization rotates in a left-hand circular pattern with one complete revolution per wave period. One particular advantage of a circularly polarized signal is that the Faraday effect does not markedly impact a circularly polarized signal. A circularly polarized signal is also less resistant to signal degradation as a result of atmospheric conditions. Moreover, there is a higher link reliability due to no risk of misalignment between the polarizations of the transmitter and receiver. Furthermore, both RHC and LHC directions can be used simultaneously on the same frequency, thus providing double the capacity for the link.

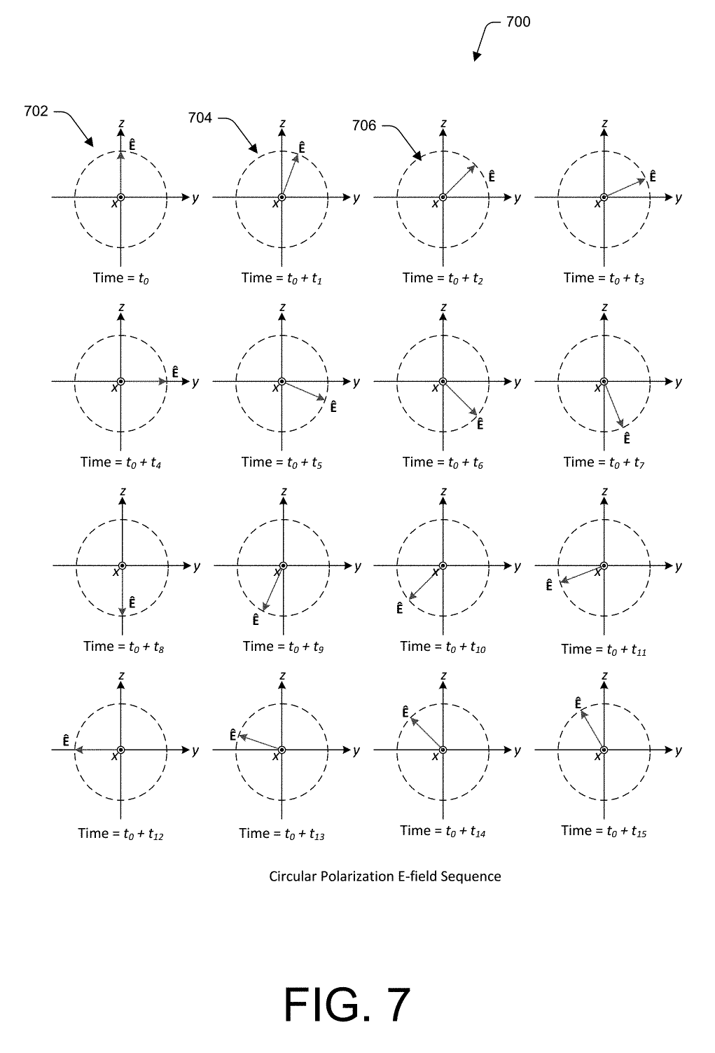

FIG. 7 illustrates the E-field sequence of a circularly polarized signal looking along the x-axis. At Time to 702, we may pick an arbitrary starting point, which is at the peak of the oscillating signal, such that the E-field Ê is at a 12 o′clock position. As the input signal to the antenna element oscillates, the output signal from the antenna transmits the signal in a rotating clockwise fashion. If we assume the illustrated signal is emanating out from the surface of the drawing in the x-direction, the clock-wise rotation indicates that a LHC signal is depicted. At time to t₀+t₁ 704, the Ê has rotated clockwise by approximately 22.5 degrees. At time to t₀+t₂ 706, the Ê has rotated clockwise by 45 degrees from the twelve o′clock position. As the signal continues, it is seen that the signal continues to rotate in a clockwise direction, completing a full revolution for each full period of the transmitted signal. The time it takes the signal to make a full resolution is dictated by the frequency, which is determined by the antenna element. As described, a patch antenna determines the frequency based upon the physical size and geometry of the conductive patch.

A radio frequency link, such as a microwave link in a telecommunications backhaul network, is generally designed to provide a robust link with minimal downtime, and in many cases, is designed to provide a reliability of 99.999%, meaning that in a years' time, the total of all outages should be less than five minutes. The incorporation of antenna elements that provide for a circularly polarized signal, and even a dual circularly polarized signal, provide a significant advantage over the currently utilized antenna systems, for many of the benefits described herein. The dual circularly polarized signals provide for increased bandwidth at the same frequency, are relatively easy to install, don't require additional dish antennas to be installed on a tower to provide a diversity scheme, and are less prone to degradation from atmospheric or Faraday effects.

The antenna 102 may be any type of antenna, such as for example, monopole, dipole, horn, planar, folded, folded inverted, planar inverted-F (PIFA), phased array, Yagi, ceramic, or printed board type (e.g., microstrip, ring microstrip, ring-slot, printed slot), or any other antenna that may broadcast or receive a circularly polarized signal. Moreover, the antenna may be utilized in any device that may communicate through a wireless radio frequency signal, and may be employed in cell phones, mobile computing devices, satellite communication systems (including satellites and earth-based stations), gaming consoles, laptop computing devices, tablet computing devices, set top boxes, and telecommunications equipment to provide subscriber coverage including cellular voice and data communications, Internet backbone communications, or telecommunication backhaul network communication links.

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A backhaul network communications link, comprising:
a first parabolic antenna configured to transmit a right-hand circular polarized radio signal at a frequency and a left-hand circular polarized radio signal at the frequency; and
a second parabolic antenna configured to receive the right-hand circular polarized radio signal at the frequency and the left-hand circular polarized radio signal at the frequency.

2. The system of claim 1, wherein the first parabolic antenna comprises a patch antenna having two feed points.

3. The system of claim 1, wherein the first parabolic antenna comprises a pair of dipole antennas.

4. The system of claim 1, wherein the frequency is within the microwave portion of the electromagnetic spectrum.

5. A polarized diversity scheme antenna system, comprising:
in a telecommunications backhaul network:
a first antenna having a first aperture; and
a first antenna element configured to broadcast a first circularly polarized signal through the aperture, and further configured to broadcast a second circularly polarized signal through the aperture, the second circularly polarized signal having an orthogonal direction of polarization to the first circularly polarized signal.

6. The antenna system of claim 5, wherein the first antenna element comprises:
a dielectric substrate;
a conductive patch disposed on the dielectric substrate, the conductive patch being generally rectangular and defining four edges;
a slot formed along at least a portion of a diagonal of the conductive patch; and
a feed point on a first edge of the conductive patch for feeding an input signal to the conductive patch.

7. The antenna system of claim 6, further comprising a second feed point on a second edge of the conductive patch, the second edge being orthogonal to the first edge.

8. The antenna system of claim 5, further comprising a second antenna having a second aperture and a second antenna element configured to receive the first circularly polarized signal and the second circularly polarized signal from the first antenna.

9. The antenna system of claim 5, wherein the conductive patch has a length that is within the range of from about 50 cm to 0.05 cm.

10. The antenna system of claim 5, wherein the antenna system is configured to transmit and receive signals within the microwave range of the electromagnetic spectrum.

11. A microwave telecommunications link, comprising:
a first antenna, the first antenna mounted stationary and having:
a reflective dish;
a first antenna element that broadcasts a first circularly polarized signal and a second circularly polarized signal, the second circularly polarized signal orthogonal to the first circularly polarized signal;
a second antenna having:
a reflective dish; and
a second antenna element that receives the first circularly polarized signal and the second circularly polarized signal.

12. The microwave telecommunication link of claim 11, wherein the second antenna is on a moveable platform.

13. The microwave telecommunication link of claim 11, wherein the first antenna is a patch antenna having two input feeds.

14. The microwave telecommunication link of claim 13, wherein the patch antenna comprises a conductive patch having a length and a width that are substantially equal.

15. The microwave telecommunication link of claim 14, wherein the two input feeds are connected to the patch antenna at a location approximately midway along the length, and approximately midway along the width.

16. The microwave telecommunication link of claim 14, wherein the length and width are within the range of from about 300 cm to 0.5 mm.

17. The microwave telecommunications link of claim 11, wherein the first antenna further comprises an antenna aperture and wherein the first circularly polarized signal and the second circularly polarized signal are broadcast through the antenna aperture.

18. The microwave telecommunications link of claim 11, wherein the first antenna is a patch antenna having a single feed and symmetric perturbation elements.

19. The microwave telecommunications link of claim 11, wherein the first antenna is a printed circular ring patch antenna.

20. The microwave telecommunications link of claim 11, wherein the first antenna broadcasts at least two de-correlated signals through a common antenna aperture.
